(19) United States
(12) Patent Application Publication
(10) Pub. No.: US 2012/0010593 A1
(43) Pub. Date: Jan. 12, 2012

(54) Method and apparatus for a strategic nerve antidote pouch

(76) Inventor: Joshua Powell, Canyon Lake, TX (US)

(21) Appl. No.: 12/803,814

(22) Filed: Jul. 7, 2010

(51) Int. Cl. A61M 5/32; A45F 5/00

(52) U.S. Cl. 604/506; 224/148.4

(57) ABSTRACT

A strategic nerve antidote pouch includes a backing, a loop panel secured to the backing, first and second straps secured to the backing, and an injector pouch member secured to the backing. The injector pouch member includes first, second, and third injector pouches that receive single nerve antidote injectors therein thereby providing the maximum allowable number of single nerve antidote injectors. The first, second, and third injector pouches provide a visual indication of the number of single nerve antidote injectors employed once a single nerve antidote injectors has been removed, injected, and discarded. The loop panel aids in preventing an accidental injection by a single nerve antidote injector, while the first and second straps along with the loop panel facilitate securing of the strategic nerve antidote pouch with a wearer's body. The strategic nerve antidote pouch further includes a protection edge mounted over a lower portion of the first, second, and third injector pouches to aid in preventing an accidental injection by a single nerve antidote injector.

### BACKGROUND OF INVENTION

1. Field of the Invention

The present invention relates to storage and administration of anti-nerve agent injectors. Specifically, the present invention provides a method and apparatus for storing and administering nerve agent antidote injectors.

2. Description of the Related Art

Emergency service or military personal responding to a chemical or biological attack need quick access to life saving equipment. Typically anti-nerve or anti-biological agents are stored in antidote injectors that are then placed in a medical service personnel's bag or vehicle. In the event of an attack, access to these life saving anti-dote injectors can be problematic as loose injectors in a bag or vehicle may be difficult to retrieve. Moreover, a person exposed to chemical or biological agent may lose their sight making it even more difficult to retrieve an injector. Furthermore, dosage of anti-nerve agents is important because a person cannot receive more than 3 doses. In chemical or biological attack, medical or military personnel exposed to nerve agents often become unconscious, which makes it difficult for other responders to determine how many dosages, if any, have been administered. Accordingly, a method and apparatus that is clearly visible, easily accessible, and allows a person to determine the dosage given or not given would be helpful.

### SUMMARY OF INVENTION

The device according to the present invention is designed to allow convenient transportation, easy access, easy identification, and safe administration of single nerve antidote injectors. The strategic nerve antidote pouch herein referred to as the SNAP was designed to be used in many different types of applications such as military and civilian. The SNAP was also designed to allow ease of transportation, ease of access, and safe administration of single nerve antidote injectors. The SNAP offers the ability for military personal and emergency workers to carry single nerve antidote injectors in a convenient way that allows the safe administration of an antidote even when a person is under the effects of a chemical or biological nerve agent. The SNAP accomplishes this task with its shape, color, and material.

The design and shape allows SNAP to be used in many different applications. The shape of the SNAP allows the pouch to stay close to the body and out of the user's way. The SNAP comes with straps that allow the pouch to be fastened to a variety of different attachments such as the ones used for military, fire, or police. The pouch straps fold back on themselves and then are snapped together making it difficult for the pouch to come unfastened. The SNAP also has an optional armband, which allows it to attach to multiple surfaces including a bare arm, a uniformed arm, a bunker jacket, or a tychem suit. The armband is adjustable, enabling it to attach to a variety of circumferences.

The design and material of the SNAP allows the color to be changed depending on application. If the user is military then a camouflage pattern can be used, if the user is a civilian emergency worker or police officer, then a brighter color can be used. The SNAP can also be fitted with a reflective strip, which renders it visible at night or in areas with little light. If stealth is required then the reflective strip can be excluded from the pouch. The material of the pouch also provides UV protection for the single nerve antidote injectors.

The injector slots of the SNAP make for safe transportation and ease of access. The purpose of the injector slots of the SNAP is to allow a gloved hand to manipulate and extract an injector without disturbing the other two injectors. The injector slots have an elastic band across the top that reinforces the hold of the injectors. This becomes important if the flap is open or if the user becomes inverted. Having slots for the injectors easily allows a person to determine how many injectors have been used. Medical procedures only allow a user to use 3 injectors on an individual. If a person is unconscious or unable to communicate, a quick glance at the pouch will tell another emergency worker how many injectors have been used and whether another anti-nerve agent can be administered to a victim.

The flap of the SNAP provides additional protection for the pouch. It provides protection to the injectors and the safety caps of the injectors. The SNAP is where the reflective strip is located. The flap also has a pull tab that allows a gloved hand to easily open the SNAP pouch.

The backing of the SNAP provides several functions. The backing maintains the rigidity of the SNAP protecting the injectors yet is still pliable and allows it to conform to an attachment surface. The backing also protects the wearer from being accidentally injected if the needle of the injector were to become exposed. This is accomplished by extending the backing past the injector slot and making the slot more narrow at the bottom. The backing also serves as an attachment point for the SNAP straps.

It is therefore an object of the present invention to provide a method and apparatus for storing and administering nerve agent antidote injectors.

Still other objects, features, and advantages of the present invention will become evident to those skilled in the art in light of the following.

### BRIEF DESCRIPTION OF THE DRAWINGS

FIG. 1 is a perspective view of the strategic nerve antidote pouch according to a preferred embodiment of my new design.

FIG. 2 is a top view thereof.

FIG. 3 is a front view thereof.

FIG. 4 is a side view thereof.

FIG. 5 is a rear view thereof.

FIG. 6 is a side view thereof.

FIG. 7 is a bottom view thereof.

### DETAILED DESCRIPTION OF THE PREFERRED EMBODIMENTS

As required, detailed embodiments of the present invention are disclosed herein; however, it is to be understood that the disclosed embodiments are merely exemplary of the invention, which may be embodied in various forms. Figures are not necessarily to scale, and some features may be exaggerated to show details of particular components or steps.

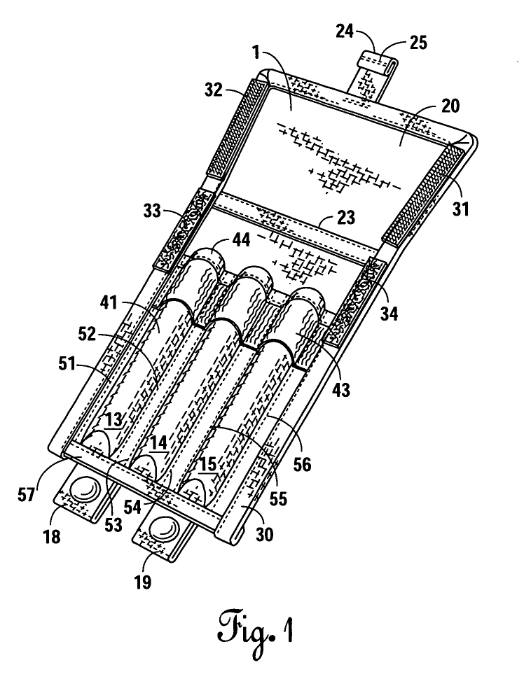

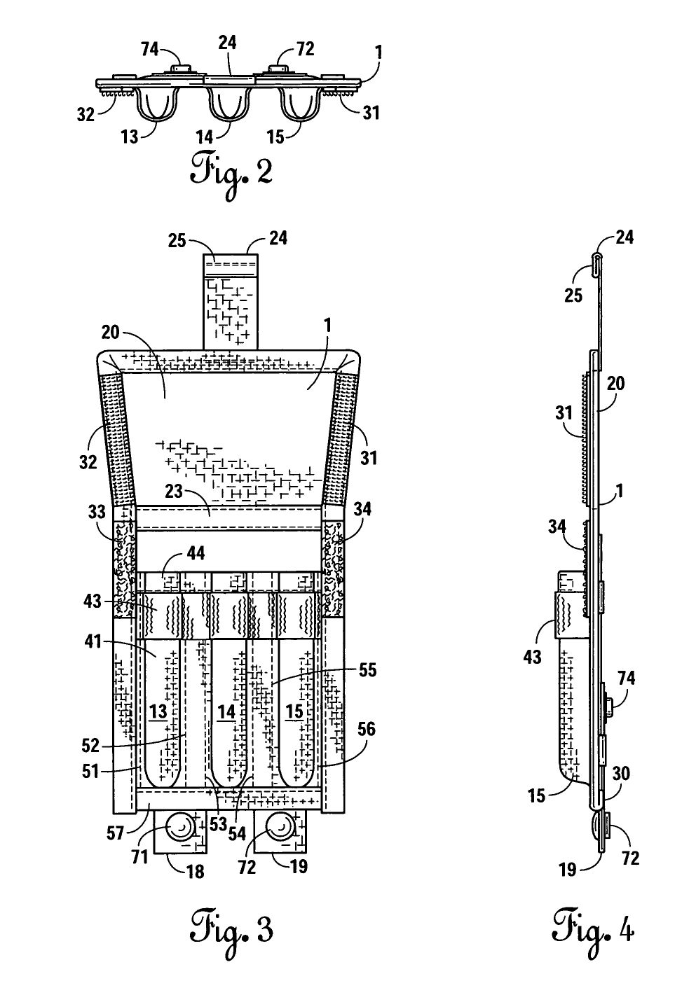

Referring to FIGS. 1-7 of the drawings, there is shown a strategic nerve antidote pouch 10 (herein referred to as a SNAP) according to a preferred embodiment of the present invention. The SNAP 10 includes a backing 1, an injector pouch member 41, a loop panel 8, straps 18 and 19, and protection edges 30 and 57.

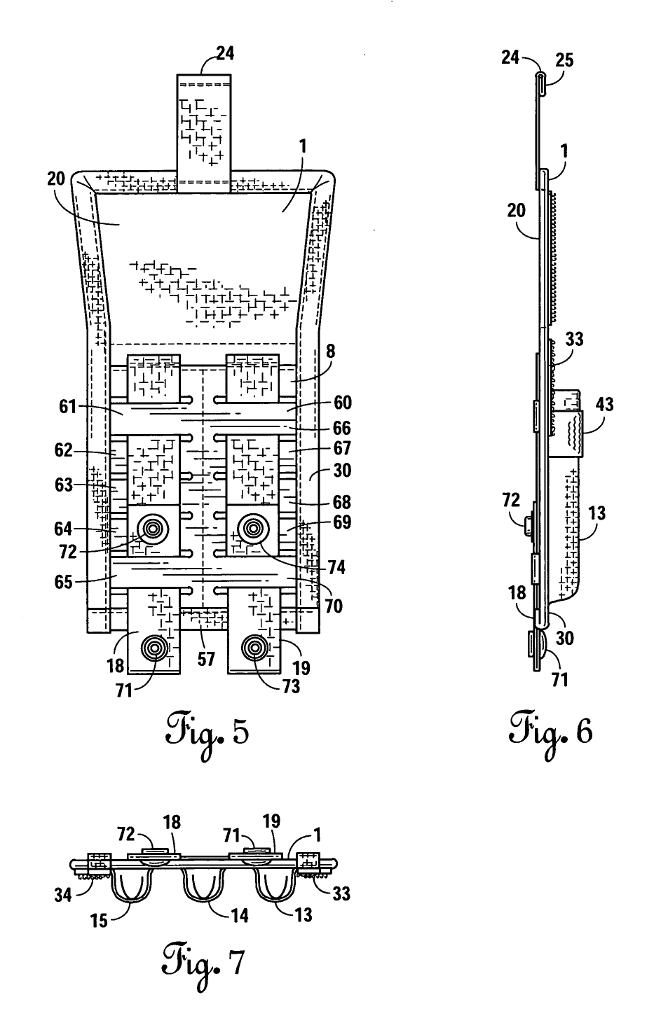

As illustrated in FIGS. 1 and 5, the backing 1 includes a front side, a back side, and a flap 20. The backing 1 provides the structure for the SNAP 10, and is an attachment point for the injector pouch member 41, the loop panel 8, the straps 18 and 19, a hinge strip 23, and the protection edges 30, 44 and 57. The material and color for the backing 1 can be changed depending upon the desired use.

As illustrated in FIGS. 1 and 3, the injector pouch member 41 includes an injector pouch sheet 40, injector pouches 13, 14, and 15, an elastic strap 43, and a protection edge 44. The protection edge 44 is a rectangular strip that wraps around the top side of the injector pouch sheet 40. It is attached to the injector pouch sheet 40 by any suitable means such as stitching or adhesive. The protection edge 44 protects the ends of injector pouches 13, 14, 15 from fraying and general wear and tear.

The injector pouch sheet 40 is placed onto the front side of the backing 1 and the injector pouch tubes 13, 14, and 15 are created by folding the injector pouch sheet 40 into cylindrical shapes that taper from the top side of injector pouch sheet 40 to the bottom side of the injector pouch sheet 40. This tapering provides a more secure fit for the injectors when they are placed into the injector pouch tubes 13, 14, and 15.

The injector pouch tube 13 is created by folding the pouch sheet 40 into a cylindrical shape that tapers from the top side of the injector pouch sheet 40 to the bottom side of the injector pouch sheet 40. After being folded into a cylindrical shape that tapers, the injector pouch tube 13 is attached to the backing 1 by stitches 51 and 52. The stitches 51 and 52 run vertically from the top side of the pouch sheet 40 to the bottom side of the pouch sheet 40.

The injector pouch tube 14 is created by folding the pouch sheet 40 into a cylindrical shape that tapers from the top side of injector pouch sheet 40 to the bottom side of injector pouch sheet 40. After being folded into a cylindrical shape that tapers, the injector pouch tube 14 is attached to the backing 1 by stitches 53 and 54. The stitches 53 and 54 run vertically from the top side of the pouch sheet 40 to the bottom side of the pouch sheet 40.

The injector pouch tube 15 is created by folding the pouch sheet 40 into a cylindrical shape that tapers from the top side of the injector pouch sheet 40 to the bottom side of the injector pouch sheet 40. After being folded into a cylindrical shape that tapers, the injector pouch tube 15 is attached to the backing 1 by stitches 55 and 56. The stitches 55 and 56 run vertically from the top side of the pouch sheet 40 to the bottom side of the pouch sheet 40.

After formation of the injector pouch tubes 13, 14, and 15, the elastic strap 43 is placed below the protection edge 43 and attached to the injector pouch sheet 40 over the injector pouch tubes 13, 14, and 15 by stitches 51-56. The elastic strap 43 helps to secure the antidote injectors within the injector pouches 13, 14, and 15.

Referring to FIG. 5, there is shown the loop panel 8 and the straps 18 and 19. The loop panel 8 and the straps 18 and 19 provide several functions to the SNAP 10. The loop panel 8 provides a support structure to SNAP 10. It also helps to prevent a person from accidentally being injected by an antidote injector. Furthermore, the loop panel 8 and the straps 18 and 19 allow the SNAP 10 to be attached to military or civil service personnel using a variety of different applications such belts, elastic bands, web gear, and the like.

The loop panel 8 has a top side, left and right side, and a bottom side. In this embodiment of the invention, the loop panel 8 is rectangular in shape with ten loops 61-70 cut out. The loop panel 8 is placed over the backing 1 on the backside of the backing 1 and secured thereto using any suitable means such as stitching or adhesive. In the present embodiment of the invention, a stitch is run across the top side of loop panel 8 and also vertically down bisecting the loop panel 8. The straps 18 and 19 are placed vertically so that they can be threaded through the loops 61-70. The straps 18 and 19 are fastened to the backing 1 using any suitable means such as stitches. The strap 18 is folded over so that the end of strap 18 is at the midpoint of strap 18. Female snap 71 is attached to the end of strap 18 and male snap 72 is attached to the midpoint of strap 18. The strap 19 is folded over so that the end of strap 19 is at the midpoint of strap 19. Female snap 73 is attached to the end of strap 18 and male snap 74 is attached to the midpoint of strap 18. The SNAP 10 is attached to a particular application by feeding straps 18 and 19 through loops 61-70 of the loop panel 8. The straps 18 and 19 are then folded back on themselves and snapped together at the snaps 71-74. In this embodiment of the invention, the loop panel 8 has ten different loops 61-70; nevertheless, more or less loops can be added depending on the application.

Referring to FIGS. 1 and 5, there is shown the protection edge 57. After the injector pouch member 41 and the loop panel 8 are attached to the backing 1, the protection edge 57 is wrapped over the bottom of the injector pouch member 40 and the loop panel 8 and is attached to the backing 1 using any suitable means such as stitching or adhesive. This overlapping helps to secure the injector pouch sheet 40 and the loop panel 8 to the backing 1. In addition, the protection edge 57 protects the bottom edges of the SNAP 10 from fraying and general wear and tear as well as prevents a person from accidentally being injected by an antidote injector. The protection edge 57 can be made of any suitable material and is attached by any suitable means such as stitching or adhesive.

Referring to FIGS. 1 and 3, there is shown a flap 20. The flap allows the SNAP 10 to close thereby protecting the antidote injectors and preventing them from falling out. The flap 20 has a front side and a backside and provides an attachment point for a hinge strip 23, a pull tab 24, and Velcro strips 31 and 32. The hinge strip 23 is placed horizontally across the front side of backing 1 and is attached thereto using any suitable means such as stitching or adhesive. The hinge strip 23 allows the flap 20 to fold consistently in the same place.

After the injector pouch member 41, the loop panel 8, the straps 18 and 19, the hinge strip 23, and the protection edge 57 are attached to the backing 1, the protection edge 30 is attached. Referring to FIGS. 1, 3 and 5, there is shown the protection edge 30. The protection edge 30 provides several useful functions to the SNAP 10. The protection edge 30 raps around the backing 1 and overlaps the injector pouch member 41, the loop panel 8, and the hinge strip 23. This overlapping helps to secure the injector pouch sheet 41, the loop panel 8 and the hinge strip 23 to the backing 1. In addition, the protection edge 30 protects the edges of the SNAP 10 from fraying and general wear and tear. The protection edge 30 can be made of any suitable material and is attached by any suitable means such as stitching or adhesive.

After the protection edge 30 is attached to the backing, the pull tab 24 and the Velcro strips 31-34 are attached. Referring to FIGS. 1, 3, and 5, there is shown the pull tab 24. The pull tab 24 is a rectangular strip which is oriented vertically and attached to the backside of the flap 20 by one of its ends using any suitable means such as stitching or adhesive. The end of the pull tab 24 that is not attached to the flap 20 is folded over and attached to itself using any suitable means such as stitching or adhesive. The folding of the pull tab 24 creates a notch 25. The notch 25 allows a user to find the flap in low light or with a gloved hand.

The Velcro strips 31 and 32 are placed on the front side of the flap 20 and over the protection edge 30. The Velcro strips 33 and 34 are placed on the front side of the backing and over the protection edge 30 so that they make contact with the Velcro strips 31 and 32 when the flap 20 is folded. The Velcro strips 31, 32, 33 and 34 are attached using any suitable means such as stitching or adhesive to help keep the flap 20 closed.

In use, a military or civilian personnel attaches the SNAP 10 to their body. This can be accomplished in several ways. The straps 18 and 19 can be fed through the loops of the loop panel 8. The straps 18 and 19 are folded back on themselves over a belt at the waist or an elastic strap. The straps 18 and 19 are snapped together. When attached to an elastic strap, the SNAP 10 may be secured to any part of the body as the elastic strap may be wrapped around a body part and secured using a suitable closing member attached to the elastic strap. The SNAP 10 accordingly can be attached at the waist or to an arm or a leg. In addition, the loop panel 8 allows the SNAP 10 be attached to military molle or civilian web gear.

In the event of a chemical or biological attack, the personnel will have three injectors readily available as the SNAP 10 is secured to their body and includes the three injector pouches 13, 14 and 15 with injectors therein. If the personnel is exposed to chemical or biological agents, the personnel opens the flap 20 of the SNAP 10, extracts an injector, administers the dose, and then discards the injector. If another dose is needed, the personnel can extract another injector and administer a dose. This can be done three times which is the maximum amount of doses that can be administered to an individual. Moreover, in the event the personnel is rendered unconscious, a further responder can easily determine the number of doses by viewing the number of injectors missing from the SNAP, thereby preventing a potential overdose. The SNAP 10 therefore imparts a method for providing safe administration of single nerve antidote injectors.

Although the present invention has been described in terms of the foregoing embodiment, such description has been for exemplary purposes only and, as will be apparent to those of ordinary skill in the art, many alternatives, equivalents, and variations of varying degrees will fall within the scope of the present invention. That scope, accordingly, is not to be limited in any respect by the foregoing description; rather, it is defined only by the claims that follow.

1. A method for an individual to carry single nerve antidote injectors, comprising:
providing a strategic nerve antidote pouch, comprising a backing and an injector pouch member secured to the backing, wherein the injector pouch member includes first, second, and third injector pouches;
placing single nerve antidote injectors into the first, second, and third injector pouches;
attaching the strategic nerve antidote pouch to an individual's body for wearing during normal activities;
removing a single nerve antidote injector from one of the first, second, and third injector pouches upon exposure to a chemical or biological agent;
injecting the removed single nerve antidote injector; and
discarding the injected single nerve antidote injector wherein an empty first, second, and third injector pouch provides a visual indication that one single nerve antidote injector has been employed.

2. The method for an individual to carry single nerve antidote injectors according to claim 1, further comprising:
removing a second single nerve antidote injector from one of the first, second, and third injector pouches upon exposure to a chemical or biological agent;
injecting the removed second single nerve antidote injector; and
discarding the injected second single nerve antidote injector wherein two empty first, second, and third injector pouches provide a visual indication that two single nerve antidote injectors have been employed.

3. The method for an individual to carry single nerve antidote injectors according to claim 2, further comprising:
removing a third single nerve antidote injector from one of the first, second, and third injector pouches upon exposure to a chemical or biological agent;
injecting the removed third single nerve antidote injector; and
discarding the injected third single nerve antidote injector wherein three empty first, second, and third injector pouches provide a visual indication that all three single nerve antidote injectors have been employed.

4. The method for an individual to carry single nerve antidote injectors according to claim 1, further comprising removing, injecting, and discarding a second and third single nerve antidote injectors as necessary.

5. A strategic nerve antidote pouch, comprising:
a backing; and
an injector pouch member secured to the backing, wherein the injector pouch member includes first, second, and third injector pouches adapted to receive single nerve antidote injectors therein thereby providing the maximum allowable number of single nerve antidote injectors, further wherein the first, second, and third injector pouches provide a visual indication of the number of single nerve antidote injectors employed once a single nerve antidote injectors has been removed, injected, and discarded.

6. The strategic nerve antidote pouch according to claim 5, further comprising:
a loop panel secured to the backing; and
first and second straps secured to the backing and adapted with the loop panel to secure the strategic nerve antidote pouch with a wearer's body.

7. The strategic nerve antidote pouch according to claim 5, wherein the loop panel aids in preventing an accidental injection by a single nerve antidote injector.

8. The strategic nerve antidote pouch according to claim 5, wherein the backing includes a flap closes the strategic nerve antidote pouch thereby protecting the single nerve antidote injectors and preventing the single nerve antidote injectors from falling out of the strategic nerve antidote pouch.

9. The strategic nerve antidote pouch according to claim 5, wherein the injector pouch member includes an elastic strap attached over the first, second, and third injector pouches to aid in securing the single nerve antidote injectors within the first, second, and third injector pouches.

10. The strategic nerve antidote pouch according to claim 5, further comprising a protection edge mounted over a lower portion of the first, second, and third injector pouches to aid in preventing an accidental injection by a single nerve antidote injector.
